(19) United States
(12) Patent Application Publication
(10) Pub. No.: US 2004/0214785 A1
(43) Pub. Date: Oct. 28, 2004

(54) Surface sanitizing compositions with improved antimicrobial performance

(75) Inventors: H. Craig Dees, Knoxville, TN (US); Eric A. Wachter, Oak Ridge, TN (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(73) Assignee: Xantech Pharmaceuticals, Inc.

(21) Appl. No.: 10/796,818

(22) Filed: Mar. 9, 2004

Related U.S. Application Data
(60) Provisional application No. 60/453,324, filed on Mar. 10, 2003.

(51) Int. Cl. A61K 31/704; A61K 31/045; A61K 31/14

(52) U.S. Cl. 514/37; 514/642; 514/717; 514/724; 514/200

(57) ABSTRACT

The present invention is directed to sanitizing compositions or preparations comprising of a combination of an alcohol-based, volatile biocide and an additional low-concentration, non-volatile antimicrobial agent. In one embodiment of the present invention, the sanitizer preparation comprises a surface sanitizing composition or preparation comprised of at least: (1) a biocide comprising a volatile alcohol at a concentration of from greater than or equal to 30% to less than or equal to 70% w/w; and (2) one or more non-volatile antimicrobial agent that is soluble in said alcohol at a concentration of from greater than or equal to 0.001% to less than or equal to 0.1% w/w.

This application claims the benefit of U.S. provisional application 60/453,324 filed Mar. 10, 2003.

### BACKGROUND OF THE INVENTION

Sanitizing agents containing alcohol and other biocidal components are commonly used to combat contamination of surfaces, such as human skin, by pathogenic biological agents, such as bacteria, fungi and viruses. Recently, the U.S. Centers for Disease Control and Prevention issued a monograph (i.e., “Guideline for Hand Hygiene in Health-Care Settings,” Morbidity and Mortality Weekly Report, Vol. 51, No. RR-16, dated Oct. 25, 2002, henceforth “MMWR/RR-16”) covering this issue; this monograph is hereby incorporated by reference in its entirety. The CDC monograph describes how the use of such sanitizing agents has evolved, and usage increased, as it has become clear that simple washing with soap and water may be inadequate:

“For generations, handwashing with soap and water has been considered a measure of personal hygiene. The concept of cleansing hands with an antiseptic agent probably emerged in the early 19^{th} century.

“In 1846, Ignaz Semmelweis observed that women whose babies were delivered by students and physicians in the First Clinic at the General Hospital of Vienna consistently had a higher mortality rate than those whose babies were delivered by midwives in the Second Clinic. He noted that physicians who went directly from the autopsy suite to the obstetrics ward had a disagreeable odor on their hands despite washing their hands with soap and water upon entering the obstetrics clinic. He postulated that the puerperal fever that affected so many parturient women was caused by “cadaverous particles” transmitted from the autopsy suite to the obstetrics ward via the hands of students and physicians. Perhaps because of the known deodorizing effect of chlorine compounds, as of May 1847, he insisted that students and physicians clean their hands with a chlorine solution between each patient in the clinic. The maternal mortality rate in the First Clinic subsequently dropped dramatically and remained low for years.

“In 1843, Oliver Wendell Holmes concluded independently that puerperal fever was spread by the hands of health personnel. Although he described measures that could be taken to limit its spread, his recommendations had little impact on obstetric practices at the time. However, as a result of the seminal studies by Semmelweis and Holmes, handwashing gradually became accepted as one of the most important measures for preventing transmission of pathogens in health-care facilities.

“In 1961, the U.S. Public Health Service produced a training film that demonstrated handwashing techniques recommended for use by health-care workers (HCWs). At the time, recommendations directed that personnel wash their hands with soap and water for 1-2 minutes before and after patient contact. Rinsing hands with an antiseptic agent was believed to be less effective than handwashing and was recommended only in emergencies or in areas where sinks were unavailable.

“In 1975 and 1985, formal written guidelines on handwashing practices in hospitals were published by CDC. These guidelines recommended handwashing with non-antimicrobial soap between the majority of patient contacts and washing with antimicrobial soap before and after performing invasive procedures or caring for patients at high risk. Use of waterless antiseptic agents (e.g., alcohol-based solutions) was recommended only in situations where sinks were not available.

“In 1988 and 1995, guidelines for handwashing and hand antisepsis were published by the Association for Professionals in Infection Control (APIC). Recommended indications for handwashing were similar to those listed in the CDC guidelines. The 1995 APIC guideline included more detailed discussion of alcohol-based hand rubs and supported their use in more clinical settings than had been recommended in earlier guidelines. In 1995 and 1996, the Healthcare Infection Control Practices Advisory Committee (HICPAC) recommended that either antimicrobial soap or a waterless antiseptic agent be used for cleaning hands upon leaving the rooms of patients with multidrug-resistant pathogens (e.g., vancomycin-resistant enterococci [VRE] and methicillin-resistant Staphylococcus aureus [MRSA]). These guidelines also provided recommendations for handwashing and hand antisepsis in other clinical settings, including routine patient care. Although the APIC and HICPAC guidelines have been adopted by the majority of hospitals, adherence of HCWs to recommended handwashing practices has remained low.” (MMWR/RR-16, pp. 1-2)

Thus, from its nascence in the mid-19th century, the use of sanitizing agents to combat pathogens has evolved into a commonplace practice by health-care workers, consumers, and others concerned about the transmission of disease.

The majority of antimicrobial agents have been designed for use in sanitizing skin of the hands, and are thus formulated as soaps or lotions, both for surgical and consumer purposes. Other such agents have been formulated for use elsewhere on the human body, including, for example in the mouth as mouthrinses.

A major component of most such antimicrobial agents is alcohol (such as ethanol or isopropanol), which exhibits potent but transient antimicrobial effects based on physical disruption of cells and denaturation of key proteins. The MMWR/RR-16 describes these effects as follows:

“The majority of alcohol-based hand antiseptics contain either isopropanol, ethanol, n-propanol, or a combination of two of these products. Although n-propanol has been used in alcohol-based hand rubs in parts of Europe for many years, it is not listed in TFM as an approved active agent for HCW handwashes or surgical hand-scrub preparations in the United States. The majority of studies of alcohols have evaluated individual alcohols in varying concentrations. Other studies have focused on combinations of two alcohols or alcohol solutions containing limited amounts of hexachlorophene, quaternary ammonium compounds, povidone-iodine, triclosan, or chlorhexidine gluconate.

“The antimicrobial activity of alcohols can be attributed to their ability to denature proteins. Alcohol solutions containing 60%-95% alcohol are most effective, and higher concentrations are less potent because proteins are not denatured easily in the absence of water . . . .

“Alcohols have excellent in vitro germicidal activity against gram-positive and gram-negative vegetative bacteria, including multidrug-resistant pathogens (e.g., MRSA and VRE), Mycobacterium tuberculosis, and various fungi. Certain enveloped (lipophilic) viruses (e.g., herpes simplex virus, human immunodeficiency virus [HIV], influenza virus, respiratory syncytial virus, and vaccinia virus) are susceptible to alcohols when tested in vitro. Hepatitis B virus is an enveloped virus that is somewhat less susceptible but is killed by 60%-70% alcohol; hepatitis C virus also is likely killed by this percentage of alcohol . . . .” (MMWR/RR-16, pp. 8-10)

In addition to alcohol, other potential components include certain agents having primarily “bacteriostatic” properties (i.e., agents, such as triclosan and benzalkonium chloride, that inhibit growth of bacteria). The known usefulness of triclosan in such capacity is discussed in MMWR/RR-16:

“Triclosan (chemical name: 2,4,4′-trichloro-2′-hydroxy-diphenyl ether) is a nonionic, colorless substance that was developed in the 1960s. It has been incorporated into soaps for use by HCWs and the public and into other consumer products. Concentrations of 0.2%-2% have antimicrobial activity. Triclosan enters bacterial cells and affects the cytoplasmic membrane and synthesis of RNA, fatty acids, and proteins. Recent studies indicate this agent's antibacterial activity is attributable to binding to the active site of enoyl-acyl carrier protein reductase.

“Triclosan has a broad range of antimicrobial activity, but it is often bacteriostatic.

Minimum inhibitory concentrations (MICs) range from 0.1 to 10 ug/mL, whereas minimum bactericidal concentrations are 25-500 ug/mL. Triclosan's activity against gram-positive organisms (including MRSA) is greater than against gram-negative bacilli, particularly P. aeruginosa. The agent possesses reasonable activity against mycobacterial and Candida spp., but it has limited activity against filamentous fungi. Triclosan (0.1%) reduces bacterial counts on hands by 2.8 log₁₀ after a 1-minute hygienic handwash. In several studies, log reductions have been lower after triclosan is used than when chlorhexidine, iodophors, or alcohol-based products are applied. In 1994, FDA TFM tentatively classified triclosan <1.0% as a Category IIISE active agent (i.e., insufficient data exist to classify this agent as safe and effective for use as an antiseptic handwash). Further evaluation of this agent by the FDA is underway. Like chlorhexidine, triclosan has persistent activity on the skin. Its activity in hand-care products is affected by pH, the presence of surfactants, emollients, or humectants and by the ionic nature of the particular formulation. Triclosan's activity is not substantially affected by organic matter, but it can be inhibited by sequestration of the agent in micelle structures formed by surfactants present in certain formulations. The majority of formulations containing <2% triclosan are well-tolerated and seldom cause allergic reactions. Certain reports indicate that providing hospital personnel with a triclosan-containing preparation for hand antisepsis has led to decreased MRSA infections. Triclosan's lack of potent activity against gram-negative bacilli has resulted in occasional reports of contamination.” (MMWR/RR-16, p. 16)

Thus, in contrast to alcohols and other biocidal components of sanitizers, bacteriostatic agents, like triclosan, are thought to suppress growth of bacteria (except when used at high concentrations, whereupon they are capable of exhibiting biocidal properties). It has also been shown in the art that triclosan is a relatively benign antimicrobial agent that is principally useful as a bacteriostat, and that topically-applied triclosan exhibits minimal penetration into human skin.

In addition to antimicrobial soaps and lotions, an additional class of antimicrobial agent is the alcohol-based sanitizer. MMRW/RR-16 notes that “these are typically an alcohol-containing preparation designed for application to the hands for reducing the number of viable microorganisms on the hands. In the United States, such preparations usually contain 60%-95% ethanol or isopropanol.” (MMWR/RR-16, p. 3)

For the purposes of this application, the following definitions are used, and are believed to be consistent with conventional usage of such terms in the field.

An antimicrobial agent is defined as a chemical compound (or preparation comprised of a mixture of two or more chemical compounds) capable of destroying or inhibiting the growth of microorganisms, such as bacteria, fungi, and viruses.

A biocide is defined as chemical compound (or preparation comprised of a mixture of two or more chemical compounds) that is immediately destructive to many different microorganisms, typically due to physical disruption of such microorganisms. Accordingly, a bacteriocidal agent is a biocide that is immediately destructive to bacteria.

A biostat is defined as chemical compound (or preparation comprised of a mixture of two or more chemical compounds) that prevents or impedes proliferation of microorganisms, typically due to interference with a critical physiological pathway of such microorganisms. Accordingly, a bacteristatic agent is a biostat that prevents or impedes proliferation of bacteria.

Persistent activity (or residual activity) is defined as prolonged or extended antimicrobial activity that prevents or inhibits the proliferation or survival of microorganisms for a period of time following application of a sanitizing agent.

A surface sanitizing composition is defined as a composition that is delivered to a surface to be sanitized, such composition comprising a liquid, an aerosol spray, or a volume of gel (such as a hydrogel) or lotion, in sufficient quantity so as to substantially cover such surface with at least a thin film of such composition. Example surfaces that may be sanitized with such composition include hard surfaces, such as counters and tabletops, telephone handsets, and bathroom fixtures, along with soft surfaces, such as human skin. Accordingly, such composition must be formulated so as to be compatible with such surfaces.

Volatile is defined as a substance that is readily vaporizable at a relatively low temperature, such as room temperature or human body temperature. Non-volatile is defined as a substance that is not volatile (i.e., not vaporizing readily at relatively low temperature).

Accordingly, it is an object of the present invention to provide new compositions, methods and preparations for antimicrobial sanitization of surfaces, including hard surfaces, soft surfaces, and human skin.

### SUMMARY OF THE INVENTION

The present invention is directed to sanitizing compositions or preparations comprising a combination of an alcohol-based, volatile biocide and an additional low-concentration, non-volatile antimicrobial agent.

In one embodiment of the present invention, the sanitizing composition or preparation comprises: (1) a biocide comprising a volatile alcohol at a concentration of from greater than or equal to 30% to less than or equal to 70% w/w; and (2) one or more non-volatile antimicrobial agent that is soluble in said alcohol at a concentration of from greater than or equal to 0.001% to less than or equal to 0.1% w/w.

In a further embodiment of the present invention, the biocide is comprised substantially of one or more of ethanol, isopropanol, and n-propanol.

In another further embodiment of the present invention, the one or more antimicrobial agent is comprised substantially of triclosan (i.e., 2,4,4′-trichloro-2′-hydroxydiphenyl ether).

In an alternate another further embodiment of the present invention, the one or more antimicrobial agent is comprised substantially of one or more of the following: benzalkonium chloride; BPI; ceftazidime; cerulenin; cetrimide; chloramphenicol; chlorhexidine; ciprofloxacin; cis-3-decynoyl-NAC; CPC; DBC; diflufenican; ethionamide; hexachlorophene; imipenem; isoniazid; isoxyl; L-16a,240; phenethyl alcohol; polymyxin B; povidone-iodine; thioenodiazaborine; thiolactomycin; thymol; and tobramycin.

In an additional embodiment of the present invention, the surface sanitizing composition is formulated as an aerosol.

In an alternate additional embodiment of the present invention, the surface sanitizing composition is formulated as a hydrogel.

In another alternate embodiment of the present invention, the surface sanitizing composition is formulated as a lotion.

In additional alternate embodiment of the present invention, the surface sanitizing composition is formulated as a liquid.

### DETAILED DESCRIPTION OF THE PRESENTLY PREFERRED EMBODIMENTS

The present invention is directed to sanitizing compositions or preparations comprising a combination of an alcohol-based, volatile biocide and a low-concentration, non-volatile antimicrobial agent. Preferably, the combination is capable of producing a potent synergistic antimicrobial effect on treated surfaces. This synergism has several key aspects. First, the volatile biocide yields an immediate kill prior to its evaporation and serves as a suitable vehicle for uniform delivery of the low-concentration antimicrobial agent. Second, the combination of biocide and antimicrobial agent yields a markedly enhanced killing of microbes (better than the efficacy of either component alone). And third, the residual non-volatile antimicrobial agent remaining on the surface after evaporation of the volatile biocide provides persistent activity against microbial recontamination of such surface.

This combination and these synergistic effects were not known and could not have been predicted by prior teachings which, in particular, have failed to present evidence of enhanced biocidal activity of such binary sanitizer preparations comprised of an alcohol-based, volatile biocide (such as ethanol or isopropanol) with an additional low-concentration, non-volatile antimicrobial agent (such as triclosan). These novel features are clearly illustrated by the following experimental data, which compare conventional sanitizer preparations with the new binary sanitizer preparations of the present invention.

### EXAMPLE 1

### Testing Sanitizer Preparations Against Gram-Positive Bacteria

Various sanitizer preparations were tested against methicillin-resistant Staphylococcus aureus (MRSA) to assess bacteriostatic and bactericidal performance against gram-positive bacteria.

Test Organism.

S. aureus was propagated from a stock collection originally isolated from the nasal pharynx of a patient at the Columbus Georgia Medical center. The organism is a highly virulent pathogen that can kill a laboratory mouse with a subcutaneous dose of less than 1×10³ staphylococci. It is also highly resistant to a broad range of antibiotics. S. aureus maintained as a frozen culture with 10% (v/v) glycerol at −80° C. was thawed and then propagated on trypticase soy broth (TSB) or agar plates at room temperature (R.T.); determinations for Most Probable Number (MPN) in given samples were performed at 37° C., using standard assay techniques.

Tested Preparations.

Tested sanitizers comprised commercially available gel products (i.e., “Brand A” and “Brand B”) from two manufacturers; these gels were comprised substantially of ethanol (60-70% w/w). Proprietary liquid preparations were also produced using standard laboratory-grade chemical reagents, including isopropyl alcohol (isopropanol), ethyl alcohol (ethanol, or EtOH), triclosan, and certain combinations thereof. Additional prototype liquid preparations (“Brand C1” and “Brand C2”) were substantially comprised of mixtures of alcohol (60-70% ethanol or isopropanol, w/w) combined with triclosan (approximately 0.04% w/w). Finally, triclosan was added to certain of the commercially available products (Brand B) to yield modified products containing triclosan at a level of approximately 0.04% w/w (“Brand B1”).

Bacteriostatic Assay.

Samples of each tested preparation were diluted into sterile TSB (1:2 or 1:10 v/v serial dilutions) across a Costar 96-well flat-bottomed tissue culture plate. Ten μL aliqouts of S . aureus inoculum (at a titer of 1×10⁷ bacteria/mL, MPN, confirmed by Colony Forming Assay, CFU) were then added to each well. The plates were incubated overnight (approximately 18 hours) at 37° C. Growth of S. aureus in each well was determined by visually observing turbidity and was confirmed using a Dynatek Microtiter plate reader at 630 nm; this allowed the bacteriostatic level (i.e., minimum inhibitory concentration, MIC) of each preparation to be readily assessed based on number of dilution steps necessary to yield positive growth.

Bactericidal Assay.

Killing of bacteria at each dilution (i.e., minimum bactericidal concentration, MBC) was determined by removing 5 μL aliquots and subculturing these on the surface of a TSB agar plate. Plates were incubated overnight at 37° C. and observed for growth. Studies were performed at 30 sec, at 5, 10, and 30 min, and at 1, 2, 4, and 8 hours after adding the challenge bacteria.

Synergism Assay.

To verify synergism of a model system (i.e., alcohol plus triclosan preparation), a separate series of tests were conducted. A triclosan stock solution was made by adding triclosan to 10 mL of either isopropanol or ethanol at 37° C.; the amount of triclosan added was determined so as to yield a final concentration of 0.01% (w/w) after dilution of the stock solution with sterile distilled water (pH 8.0, 45° C., yielding a final alcohol concentration of 10% v/v). Temperature of the mixture was maintained at 40° C. during subsequent serial dilution into room temperature TSB (using microwell plates, as described supra). Serial dilutions of 1:2 and 1:10 (v/v) were made to yield a final dilution of 1:10¹⁰. Ethanol and isopropyl alcohol (60% v/v in water) were similarly serially diluted, alone or added in combination with triclosan. The resultant dilutions were challenged by addition of 10 μL of S. aureus (1×10⁵-10⁶ bacteria/mL). Plates were incubated overnight at 37° C. and growth of the bacterium confirmed by turbidity in the wells. Wells were subcultured on TSB plates (as described supra) for determination of bactericidal activity.

Assay Results.

Results of these assays are summarized in Table 1, which illustrates a number of important observations. Various alcohols (i.e., EtOH, isopropanol, and the EtOH-based gels), alone in standard unary preparation, exhibit extremely limited bacteriostatic performance. Conversely, triclosan alone exhibits marked bacteriostatic performance, even when highly diluted. The addition of triclosan to alcohol, comprising a binary preparation, yields bacteriostatic performance that is comparable to triclosan alone. Such additive response is expected, since alcohol has limited bacteriostatic properties upon dilution while triclosan is known to have a wide range of bacteriostatic activity. Accordingly, the bacteriostatic effects of each component in such preparations are additive. In contrast to these results, the synergistic bactericidal response that is noted for such binary preparations is completely without precedent. For instance, neither triclosan nor any unary alcohol preparation exhibited bactericidal activity when diluted by more than a factor of 10 (i.e., 1:10¹); combination of any of these alcohols with triclosan in a binary preparation exhibited greatly enhanced bactericidal activity, as evidenced by the markedly enhanced resistance of these preparations to the effects of dilution. Specifically, whereas dilutions greater than 10-fold of triclosan or alcohol were not bactericidal upon challenge with 1×10⁴ MRSA, the binary preparations exhibited synergistic bactericidal activity even when diluted 1000-fold.

Additional Observations.

Brief exposure of MSRA to the various preparations demonstrated that any bactericidal effect occurred within 30 seconds (i.e., within the minimum contact time tested), with no additional effect for exposures up to 8 hours.

TABLE 1
Bacteriostatic (i.e., MIC) and bactericidal (i.e., MBC)
performance of sanitizer preparations against MRSA.
Various preparations diluted (v/v) in water, then
challenged with a dose of 10 μL of
1 × 10⁵-1 × 10⁷ bacteria/mL
(i.e., 1 × 10³-1 × 10⁵ MRSA).
Reported values are maximum dilutions exhibiting bacteriostatic
or bactericidal performance, respectively.

| | | | | MBC @ challenge | |
|---|---|---|---|---|---|
| | MIC @ challenge level | | | level | |
| | 1 × 10⁵ | 1 × 10⁴ | 1 × 10³ | 1 × 10⁵ | 1 × 10⁴ |
| Preparation | MRSA | MRSA | MRSA | MRSA | MRSA |
| Triclosan | | | 1:10¹⁰ | | ≦1:10¹ |
| EtOH | | 1:4 | 1:10¹ | | 1:10¹ |
| Isopropanol | | 1:4 | 1:10¹ | | 1:10¹ |
| EtOH Gel (Brand A) | 1:4 | | | <1:2 | |
| EtOH Gel (Brand B) | 1:4 | | | | |
| EtOH Gel + | 1:10⁶ | | | | |
| Triclosan (Brand B1) | | | | | |
| EtOH + | 1:10⁷ | | 1:10¹⁰ | 1:10² | 1:10³ |
| Triclosan (Brand C1) | | | | | |
| Isopropanol + | 1:10⁷ | | 1:10¹⁰ | 1:10² | 1:10³ |
| Triclosan (Brand C2) | | | | | |

Surface Studies.

To assess residual effectiveness of the various sanitizer preparations against methicillin-resistant Staphylococcus aureus (MRSA) on surfaces, each preparation was applied to a sterile surface, the sanitized surface was allowed to dry, the dried surface was contaminated with S. aureus, and the resultant contaminated surface was then sampled over a period of hours to assess bacteriostatic and bactericidal performance.

One hundred μL of a given test preparation were added to a sterile well (1.2 cm) of a Lab-Tek II chamber slide (4 well); the material was evenly distributed over the surface and allowed to dry for 30 seconds. Gentle wiping with a sterile cotton swab removed residual materials. One well treated with ethanol and triclosan preparation was subsequently washed 5 times with 2 mL each of sterile distilled water (to assess resistance of the treated surface to water exposure). Another well was kept untreated to serve as a control. Two hundred μL of S. aureus (at a titer of 1×10⁷ bacteria/mL, or 2×10⁶ bacteria) were then added to each well. At various time intervals following this contamination, 10-μL aliqouts of this culture media were removed and serially diluted to determine MPN of surviving bacteria. Subcultures of these dilutions were made to determine bactericidal activity.

Surface Results.

The results in Table 2 demonstrate that alcohol alone (i.e., Brand A and Brand B gels) exhibits no residual bacteriostatic or bactericidal effect on surfaces. Once it evaporates, it does not inhibit contamination by and growth of MRSA. In contrast, surfaces treated with binary preparations comprised of alcohol and triclosan resisted contamination at all times sampled (up to 8 hr). This persistent activity was unaffected by multiple rinsing of the treated surface with water, illustrating that the effect is quite robust. Addition of triclosan to a commercial gel sanitizer (i.e., Brand B1) afforded comparable protection against surface contamination. Short-term effects are further demonstrated by the data in Table 3, which show that all bacteria are killed within 30 s on surfaces treated with a binary alcohol and triclosan preparation. Taken together, the data in Tables 2 and 3 illustrate that the bacteriostatic and bactericidal effects of binary alcohol and triclosan preparations are both rapid and persistent, and are markedly superior to unary preparations (such as alcohol alone).

TABLE 2
Persistent activity against surface contamination with
MRSA following treatment with sanitizer. MPN of viable
bacteria present on the test surface was determined at
each elapsed time (since treatment of surface with sanitizer).
Challenge dose of 2 × 10⁶ MRSA/mL.

| Surface | MPN (at Elapsed Time) | | | | | |
|---|---|---|---|---|---|---|
| Treatment (pre-Challenge) | 0 | 0.25 hr | 1 hr | 2 hr | 4 hr | 8 hr |
| No Treatment | 10⁶ | 10⁶ | 10⁶ | 10⁷ | 10⁷ | 10⁷ |
| EtOH + Triclosan | 0 | 0 | 0 | 0 | 0 | 0 |
| EtOH + Triclosan (then rinsed | 0 | 0 | 0 | 0 | 0 | 0 |
| with H₂O) | | | | | | |
| EtOH Gel (Brand A) | 10⁶ | 10⁶ | 10⁶ | 10⁷ | 10⁷ | 10⁷ |
| EtOH Gel (Brand B) | 10⁶ | 10⁶ | 10⁶ | 10⁶ | 10⁶ | 10⁶ |
| EtOH Gel + | 0 | 0 | 0 | 0 | 0 | 0 |
| Triclosan (Brand B1) | | | | | | |

TABLE 3
Demonstration of short-term effectiveness of
sanitizer residue against surface contamination with MRSA.
MPN determined upon exposure of surface to MRSA
at each elapsed time (since treatment of surface with sanitizer).
Challenge dose of 2 × 10⁶ MRSA/mL.

| | MPN (at Elapsed Time) | | |
|---|---|---|---|
| Surface Treatment (pre-Challenge) | 30 s | 5 min | 10 min |
| No Treatment | 10⁶ | 10⁶ | 10⁶ |
| EtOH + Triclosan | 0 | 0 | 0 |

### EXAMPLE 2

### Testing Sanitizer Preparations Against Gram-Negative Bacteria

Various sanitizer preparations were tested against antibiotic-resistant Escherichia coli (E. coli) to assess bacteriostatic and bactericidal performance against gram-negative bacteria.

Test Organism.

E. coli(P+) was propagated from a stock collection originally isolated from a urine culture of a patient at the Columbus Georgia Medical center. E. coli maintained as a frozen culture with 10% (v/v) glycerol at −80° C. was thawed and then propagated on TSB or agar plates at room temperature; determinations for Most Probable Number (MPN) in given samples were performed by serially diluting 1:10 (v/v) in a 96 well microtiter plate followed by incubation overnight at 37° C.

Test Preparations.

Tested preparations were as described supra for gram-positive assays.

Bacteriostatic Assay.

Samples of each tested preparation were diluted into sterile TSB (1:2 or 1:10 v/v serial dilutions) across a Costar 96-well flat-bottomed tissue culture plate, as described supra for similar tests using gram-positive bacteria. Ten μL aliqouts of E. coli inoculum (at a titer of 1×10⁷ bacteria/mL, MPN) were then added to each well. The plates were incubated overnight (approximately 18 hours) at 37° C. Growth of E. coli in each wells was determined by visually observing turbidity and was confirmed using a Dynatek Microtiter plate reader at 630 nm; this allowed the bacteriostatic level of each preparation to be readily assessed based on number of dilution steps necessary to yield positive growth.

Bactericidal Assay.

Killing of bacteria at each dilution (bactericidal level) was determined by removing 5 μL aliquots and subculturing these on the surface of a TSB agar plate, as described supra for similar tests using gram-positive bacteria. Plates were incubated overnight at 37° C. and observed for growth. Studies were performed at 30 sec, at 5, 10, and 30 min, and at 1, 2, 4, and 8 hours after adding the challenge bacteria.

Assay Results.

Results of these assays are summarized in Table 4, which illustrates a number of important observations. Alcohol alone (i.e., EtOH-based gels) exhibited extremely limited bacteriostatic performance against gram-negative bacteria. Addition of triclosan to alcohol, comprising a binary preparation (i.e., Brand B1 and Brand C1), yielded potent bacteriostatic performance. Both of these results are comparable to those demonstrated supra against gram-positive bacteria, and are consistent with the known properties of such agents when used singly. In contrast to these MIC results, the synergistic bactericidal response that is demonstrated for the binary preparations is, as in the case of gram-positive bacteria, completely without precedent. For instance, triclosan is not purported to have significant bactericidal properties at the low concentrations used in this assay. Nonetheless, the combination of alcohol with triclosan in a binary preparation exhibited greatly enhanced bactericidal activity relative to alcohol alone, as evidenced by the markedly enhanced resistance of binary preparations to the effects of dilution. Such synergistic behavior is comparable to that demonstrated supra against gram-positive bacteria.

Additional Observations.

Brief exposure of gram-negative bacteria to the various preparations demonstrated that any bactericidal effect occurred within 30 seconds (i.e., within the minimum contact time tested), with no additional effect for exposures up to 8 hours. Such response was comparable to that demonstrated supra against gram-positive bacteria.

TABLE 4
Bacteriostatic (MIC) and bactericidal (MBC) performance
against E. coli. Various preparations diluted (v/v) in water,
then challenged with a challenge dose of
10 μL of 1 × 10⁶-1 × 10⁷
bacteria/mL (i.e., 1 × 10⁴-1 × 10⁵ E. coli). Reported values are
maximum dilutions exhibiting bacteriostatic or bactericidal
performance, respectively.

| | MIC @ | MBC @ challenge |
|---|---|---|
| | challenge level | level |
| Preparation | 1 × 10⁴ E. coli | 1 × 10⁵ E. coli |
| EtOH Gel (Brand A) | 1:2 | <1:2 |
| EtOH Gel (Brand B) | 1:2 | |
| EtOH Gel + Triclosan (Brand B1) | 1:10⁶ | 1:10³ |
| EtOH + Triclosan (Brand C1) | 1:10⁶ | 1:10³ |

Surface Studies.

Twenty five microliters (25 μL) of active E. coli culture (10⁷ bacterial/mL) were evenly spread over 1-cm diameter circular patches of human skin (on the forearm) and allowed to dry briefly. Twenty five microliters (25 μL) of santitizer were then spread over each area. One circle treated with bacteria was left untreated to as a control. One minute after sanitizer application a sterile tube containing 3 mL of TSB was placed on the circle and inverted 3 times to wash bacteria from the test site. The bacteria in each wash solution were collected by vacuum filtration onto the surface of a sterile 0.22 micron filter (Nalgene Analytic 150 mL filter unit). The filters were aseptically removed and placed onto the surface of a McConky's agar plate, then incubated overnight at 37° C. This sampling procedure was repeated at each test site at various elapsed times (up to 6 hr post-sanitization). The resultant incubated plates were observed for formation of E. coli colonies (CFUs).

Surface Results.

The results in Table 5 demonstrate that alcohol alone (i.e., Brand A and Brand B gels) exhibit transient bactericidal effects against gram-negative bacterial contamination of human skin, but that the effect is non-persistent (i.e., no bacteriostatic effect is noted since residual E. coli levels mount as time elapses). This is comparable to similar surface effects noted for gram-positive bacteria, which were not suppressed once the alcohol of these preparations evaporated. In contrast, surfaces treated with binary preparations comprised of alcohol and triclosan not only exhibited immediate bactericidal effects, but these preparations also exhibited persistent bacteriostatic activity at all times sampled (up to 6 hr). This persistent activity is especially notable since no extraordinary steps were taken to prevent further bacterial contamination of the treated sites post-sanitization. Thus, as demonstrated for gram-positive bacteria, these data illustrate that the bacteriostatic and bactericidal effects of a binary alcohol and triclosan preparation against gram-negative bacteria are both rapid and persistent, and are markedly superior to unary preparations (such as alcohol alone).

TABLE 5
Colony Forming Units of viable E. coli on human skin at
various time points post-sanitization.

| | CFUs (at Elapsed Time) | | | |
|---|---|---|---|---|
| Surface Treatment (pre-Challenge) | 1 min | 1 hr | 2 hr | 6 hr |
| No Treatment | TNC* | 15 | 25 | 97 |
| EtOH Gel (Brand A) | 2 | 13 | 25 | 78 |
| EtOH Gel (Brand B) | 3 | 15 | 21 | 71 |
| EtOH Gel + Triclosan (Brand B1) | 0 | 0 | 0 | 0 |
| EtOH + Triclosan | 2 | 0 | 0 | 0 |

*Too Numerous to Count (TNC)

Relevance of Experimental Data with Bacteria

As noted supra, it is known that a sanitizer preparation containing triclosan has bacteriostatic properties. For example, the MIC for triclosan is known to range from 0.1 to 10 μg/mL (i.e., see MMWR/RR-16, p. 16). In contrast, the synergistic bactericidal properties of the binary sanitizer preparation, comprised of a volatile aliphatic alcohol (such as ethanol or isopropanol) and a low-concentration, non-volatile antimicrobial agent (i.e., a bacteriostat, such as triclosan), of the present invention is not known. For example, while antimicrobial activity of a number of binary preparations, including one or more containing 60-70% alcohol plus triclosan at a concentration of greater than or equal to 0.25% has been noted (see Jones et al., “Triclosan: A Review of Effectiveness and Safety in Health Care Settings,” Am. J. Infect. Control, vol. 28, p. 191, 2000), the prior teachings fail to note any potential synergism of such components, particularly at low triclosan concentrations. Further, other references (see Johnson et al. “Comparative Susceptibility of resident and transient hand bacteria to parachloro-meta-xylenol and triclosan,” J. Appl. Microbiol., vol. 93, p. 339, 2002) report relatively high MBC values for triclosan: 7.5 mg/L against S. aureus, and 1.3 mg/L against E. coli.

In contrast to this reference, the data in Tables 1 and 4 of the present application show that MBC levels for a binary preparation consisting of alcohol and triclosan of the present invention are equivalent to less than 0.4 mg/L against both S. aureus and E. coli (i.e., 0.04% triclosan in 60-70% alcohol, when diluted 1:10³, is bactericidal against a challenge dose of 1×10⁴ MRSA and 1×10⁵ E. coli). The relevance of this synergism is further confirmed by comparing the performance of the unary preparations against that of the binary preparations for the particular strains of bacteria used in the present experiments. For example, the data in Table 1 show that the binary preparations are approximately 100-fold more bactericidal against S. aureus than would be predicted based on an additive effect for the individual antimicrobial agents. Similarly, the data in Table 4 show a greater than 100-fold increase in bactericidal activity E. coli relative to that predicted based on such an additive effect. Thus, the binary sanitizer preparations substantially comprised of alcohol and a low-concentration, non-volatile antimicrobial agent, such as triclosan, of the present invention exhibit unanticipated bactericidal synergy.

The synergy demonstrated by the present invention not only enables sanitizer preparations to exhibit improved bactericidal activity, but allows novel formulation of efficacious preparations using bacteriostatic component concentrations well below that predicted based on prior teachings. More specifically, the data presented in the present application demonstrate that such preparations will be efficacious surface sanitizers even when bacteriostat concentrations are at levels of 0.1% and lower. Even at such concentrations, the noted synergistic effect increases the resilience of such preparations to dilution (i.e., due to their inherent extended range of efficacy). Moreover, the use of such reduced levels minimizes potential for irritation of skin or other damage to sanitized surfaces, and reduces cost of manufacture.

### EXAMPLE 3

### Testing Sanitizer Preparations Against Viruses

Various sanitizer preparations were tested against Feline Enteric Cornavirus (FECV, which is of the same viral class as the coronavirus that causes SARS) in order to assess antiviral performance.

Test Organism.

FECV was purchased from the American Type Culture Collection (ATCC). CRFK cells to propagate the virus for stock virus and to titer virus were also obtained from ATCC. CRFK cells were grown using Dubecco's Modified Eagles medium (DMEM) with F-12 Ham's nutrients (Sigma); DMEM was supplemented with ampicillin, gentamycin, 7% Fetal Bovine serum, Hepes, sodium bicarbonate and glutamate. CRFK cells were maintained and propagated at 37° C. in a humidified atmosphere with 5% CO₂ Virus titration on CRFK cells was performed under the same conditions. Cells were grown in 25 cm² or 150 cm² Corning Tissue culture flasks. Virus titration was performed using Costar 96-well cell culture cluster.

Tested Preparations.

Tested sanitizers comprised of a commercially available gel product (i.e., “Brand D”) which was comprised substantially of ethanol (ca. 60% w/w). A proprietary liquid test preparation (i.e., “EtOH+Triclosan”) was produced using standard chemical reagents, including ethyl alcohol (60% w/w) and triclosan (0.04% w/w).

Effectiveness Testing.

Sterile Lab-Tek II chamber slides (1 well ) were used to demonstrate the effectiveness of viral killing on a surface.

Immediate Effectiveness.

FECV (10⁵TCID, Tissue culture infectious dose) was spread on the surface of the glass chamber slide in a circle approximately 1 cm in diameter and allowed to dry (approximately 5-7 minutes). Approximately 100 μL of one of the test preparations was then spread over the area containing FECV. Approximately 30 sec later, 200 μL of DMEM was added to the treated area. The medium was aspirated and transferred to a well of a 96-well microtiter tissue culture plate with a confluent monolayer of CRFK. CRFK cells in the microtiter plates were incubated for 72 hours and observed. Death of CRFK cells within this incubation period indicated presence of viable virus.

Residual Effectiveness.

Approximately 100 μL of one of the test preparations was evenly distributed over a 1 cm diameter surface on the Lab-Tek slides and allowed to remain on the surface until dry (i.e., for approximately 5 minutes). Treated slides were washed 5 times with 2 mL of sterile distilled water. The slides were then incubated at room temperature for 8 hours. After 8 hours of incubation, 10⁵ TCID of FECV was added to the 1 cm area and allowed to dry (approximately 5-8 minutes). After drying, virus infectivity was recovered and titered as described for immediate effectiveness studies.

Assay Results.

Results of these assays are summarized in Table 6, which illustrates a number of important observations. The alcohol-based preparations (i.e., Brand D and EtOH+Triclosan) exhibited comparable antiviral performance against coronavirus, killing over 99.9% of viruses on tested surfaces. However, only the binary preparation (i.e., EtOH+Triclosan) yielded residual effectiveness. This residual effectiveness is all the more remarkable given that the surfaces were rinsed 5 times with water between treatment and viral challenge. Thus, as in the case of antibacterial properties, the binary preparation yields superior protection, particularly after volatilization of the alcohol component.

TABLE 6
Antiviral performance of sanitizer preparations against FECV.
Preparations were applied (a) to a contaminated
surface (immediate results) or (b) to a sterile surface that was
subsequently contaminated 8 hours after
application (residual results).

FECV Titer

| Preparation | TCID | Immediate | Residual |
|---|---|---|---|
| No Treatment | 10⁵ FECV | 10⁵ | 10⁵ |
| Brand D | 10⁵ FECV | 10¹ | 10⁵ |
| EtOH + Triclosan | 10⁵ FECV | 10¹ | 10¹ |

Advanced Surface Sanitizing Preparations.

The novel sanitizer preparations of the present invention comprise, preferably, a binary sanitizer preparation itself comprised substantially of a volatile aliphatic alcohol (such as ethanol or isopropanol, at a concentration of between approximately 30% and 70%), and a low-concentration, non-volatile antimicrobial agent (i.e., a bacteriostat, such as triclosan). As explained below, the present invention meets all of the following parameters which are relevant to selection of the antimicrobial component:

not be substantially absorbed by human skin;

have low toxicity to humans and a known safety profile;

afford known bacteriostatic properties at low concentrations;

provide synergistic biocidal properties when used in conjunction with alcohol;

be non-soluble in water, thus facilitating persistent activity;

be of moderate- to low-cost; and

be chemically and physically stable.

Antimicrobial agents that fit these criteria will afford safe, effective, persistent, stable, and low cost sanitizers, as taught herein.

Skin Absorption.

It has been shown that topically-applied triclosan exhibits minimal penetration into human skin. Thus, triclosan fits this criterion.

Toxicity and Safety.

The low toxicity and safety of triclosan are well established.

Bacteriostatic Properties.

The bacteriostatic properties of triclosan are also well established.

Synergistic Properties.

The synergistic biocidal properties of triclosan, when used in conjunction with alcohol, although previously unknown, have been demonstrated herein.

Water Solubility.

It is preferred that the antimicrobial agent be substantially insoluble in water. This property will assure that residue of such agent will be resistant to inadvertant removal resulting from incidental water contact, such as rinsing of surface, and thereby increase persistent activity of the preparation. Triclosan, the example antimicrobial agent described in detail supra, is substantially insoluble in water, making it an ideal match for this criterion.

Cost and Stability.

Triclosan is known to be of moderate cost and high stability.

The special combination of properties of triclosan allow it to be used at a concentration of less than or equal to 0.1%. Such low levels will not leave a visible film or other apparent residue, further enhancing the properties of the sanitizer preparation. Moreover, few other antimicrobial agents exhibit significant skin irritation at such levels.

The preferred sanitizing compositions or preparations of the present invention, comprised substantially of alcohol and an antimicrobial agent, can be formulated in any of a number of physical forms, including: liquid; semi-solid, such as a gel, hydrogel or lotion; and as an aerosol.

It is further preferred that these sanitizing compositions or preparations comprise a combination of an alcohol-based, volatile biocide and an additional low-concentration, non-volatile antimicrobial agent.

In one embodiment of the present invention, these sanitizing compositions or preparations comprise: (1) a biocide comprising a volatile alcohol at a concentration of from greater than or equal to 30% to less than or equal to 70% w/w; and (2) one or more non-volatile antimicrobial agent that is soluble in said alcohol at a concentration of from greater than or equal to 0.001% to less than or equal to 0.1% w/w.

In a further embodiment of the present invention, the biocide is comprised substantially of one or more of ethanol, isopropanol, and n-propanol.

In another further embodiment of the present invention, the one or more antimicrobial agent is comprised substantially of triclosan (i.e., 2,4,4′-trichloro-2′-hydroxydiphenyl ether).

In an alternate another further embodiment of the present invention, the one or more antimicrobial agent is comprised substantially of one or more of the following: benzalkonium chloride; BP1; ceftazidime; cerulenin; cetrimide; chloramphenicol; chlorhexidine; ciprofloxacin; cis-3-decynoyl-NAC; CPC; DBC; diflufenican; ethionamide; hexachlorophene; imipenem; isoniazid; isoxyl; L-16a,240; phenethyl alcohol; polymyxin B; povidone-iodine; thioenodiazaborine; thiolactomycin; thymol; and tobramycin.

In an additional embodiment of the present invention, these sanitizing compositions or preparations are formulated as an aerosol.

In an alternate additional embodiment of the present invention, these sanitizing compositions or preparations are formulated as a hydrogel.

In another alternate embodiment of the present invention, these sanitizing compositions or preparations are formulated as a lotion.

In an additional alternate embodiment of the present invention, these sanitizing compositions or preparations are formulated as a liquid.

This description has been offered for illustrative purposes only and is not intended to limit the invention of this application.

What is claimed and desired to be protected by Letters Patent is set forth in the appended claims.

We claim:

1. A sanitizing composition comprising:
an alcohol-based volatile biocide; and
a low-concentration, non-volatile antimicrobial agent.

2. The sanitizing composition of claim 1 wherein said the biocide is comprised substantially of at least one alcohol selected from the group consisting of ethanol, isopropanol, and n-propanol.

3. The sanitizing composition of claim 1 wherein said antimicrobial agent is comprised substantially of triclosan (i.e., 2,4,4′-trichloro-2′-hydroxydiphenyl ether).

4. The sanitizing composition of claim 1, wherein said antimicrobial agent is comprised substantially of at least one agent selected from the group consisting of benzalkonium chloride; BP 1; ceftazidime; cerulenin; cetrimide; chloramphenicol; chlorhexidine; ciprofloxacin; cis-3-decynoyl-NAC; CPC; DBC; diflufenican; ethionamide; hexachlorophene; imipenem; isoniazid; isoxyl; L-16a,240; phenethyl alcohol; polymyxin B; povidone-iodine; thioenodiazaborine; thiolactomycin; thymol; and tobramycin.

5. The sanitizing composition of claim 1 wherein said sanitizing composition is formulated as an aerosol.

6. The sanitizing composition of claim 1 wherein the sanitizing composition is formulated as a hydrogel.

7. The sanitizing composition of claim 1 wherein the sanitizing composition is formulated as a lotion.

8. The sanitizing composition of claim 1 wherein the sanitizing composition is formulated as a liquid.

9. A sanitizing composition comprising:
a biocide comprising a volatile alcohol at a concentration of from greater than or equal to 30% to less than or equal to 70% w/w; and
at least one non-volatile antimicrobial agent that is soluble in said alcohol at a concentration of from greater than or equal to 0.001% to less than or equal to 0.1% w/w.

10. The sanitizing composition of claim 9 wherein said the biocide is comprised substantially of at least one alcohol selected from the group consisting of ethanol, isopropanol, and n-propanol.

11. The sanitizing composition of claim 9 wherein said antimicrobial agent is comprised substantially of triclosan (i.e., 2,4,4′-trichloro-2′-hydroxydiphenyl ether).

12. The sanitizing composition of claim 9 wherein said antimicrobial agent is comprised substantially of at least one agent selected from the group consisting of benzalkonium chloride; BP 1; ceftazidime; cerulenin; cetrimide; chloramphenicol; chlorhexidine; ciprofloxacin; cis-3-decynoyl-NAC; CPC; DBC; diflufenican; ethionamide; hexachlorophene; imipenem; isoniazid; isoxyl; L-16a,240; phenethyl alcohol; polymyxin B; povidone-iodine; thioenodiazaborine; thiolactomycin; thymol; and tobramycin.

13. The sanitizing composition of claim 9 wherein said sanitizing composition is formulated as an aerosol.

14. The sanitizing composition of claim 9 wherein the sanitizing composition is formulated as a hydrogel.

15. The sanitizing composition of claim 9 wherein the sanitizing composition is formulated as a lotion.

16. The sanitizing composition of claim 9 wherein the sanitizing composition is formulated as a liquid.

17. Use of an alcohol-based volatile biocide and at least one low-concentration, non-violate microbial agent in the preparation of a sanitizing composition for consumer and surgical use.
